(19) United States
(12) Patent Application Publication
(10) Pub. No.: US 2024/0131237 A1
(43) Pub. Date: Apr. 25, 2024

(54) MOBILE INTERFACE CART FOR USE IN A DIALYSIS SYSTEM

(71) Applicant: Fresenius Medical Care Holdings, Inc., Waltham, MA (US)

(72) Inventors: Kenneth Nelson, Aurora, CO (US); Juliet Hildreth, Pittsford, NY (US); Steven Michael Schmitt, Muskegon, MI (US)

(21) Appl. No.: 17/972,934

(22) Filed: Oct. 24, 2022

(51) Int. Cl. A61M 1/16; A61G 12/00; A61M 1/00

(52) U.S. Cl. CPC A61M 1/1603; A61G 12/001; A61M 1/84; A61M 2205/18; A61M 2205/3331

(57) ABSTRACT

A mobile interface cart including a storage tank for receiving effluent from a dialysis (e.g., hemodialysis) procedure and a pump for pumping the effluent from the storage tank to an external waste drain located in the treatment center. In use, the pump enables the effluent to be moved over greater distances and/or around obstacles thereby eliminating, or at least minimizing, the need to construct expensive and time-consuming waste drains in the treatment center.

### FIELD OF THE DISCLOSURE

The disclosure generally relates to dialysis machines and systems, and more particularly to a mobile interface cart arranged and configured to pump effluent to a waste drain.

### BACKGROUND

Dialysis machines are known for use in the treatment of renal disease. The two principal dialysis methods are hemodialysis (HD) and peritoneal dialysis (PD). During PD, a dialysis machine, such as a PD machine, may include one or more containers (e.g., bags) containing a fluid (e.g., dialysis solution) for patient infusion. In addition, a PD machine may include a removable and/or replaceable cartridge or cassette attached to one or more fluid lines for pumping fluid to and from a patient. During PD, for example, one or more fluid lines are inserted into an abdomen of a patient for flowing fresh dialysate and removing used dialysate, waste, and excess fluid (collectively referred to herein as spent dialysis and/or spent dialysate without the intent to limit). During a treatment operation, the PD machine pumps fluid, e.g., fresh and spent dialysate, to and from a patient. For example, the PD machine may pump dialysate from one or more dialysate bags and into the patient's abdomen. The dialysate may pass through a heating chamber to heat the dialysate to a predetermined temperature prior to inserting the dialysate into the machine. Next, the dialysis machine may remove the spent dialysate from the patient's abdomen. That is, after the dialysate has been inserted into the patient's abdomen and a prescribed amount of urea, creatine, water, and other waste matter (e.g., effluent) have been removed from the patient, the spent dialysate must be discarded.

During HD, a hemodialysis system is arranged and configured to, inter alia, pump a patient's blood through a dialyzer. In addition, the hemodialysis system includes a HD machine and one or more containers containing a fluid (e.g., a dialysate), which during use is also pumped through the dialyzer of the HD system simultaneously with the patient's blood. A semi-permeable membrane in the dialyzer separates the blood from the dialysate within the dialyzer and allows diffusion and osmosis exchanges to take place between the dialysate and the blood. Thus arranged, in use, the patient's blood is cleaned or filtered. That is, as will be readily appreciated by one of ordinary skill in the art, a hemodialysis system includes a HD machine including a dialysate flow path, an extracorporeal blood flow path, and a dialyzer. In addition, the hemodialysis system may include any number of additional components needed to manage, sense, etc. the flow of dialysate and/or blood through the system. In use, the HD machine and the dialysate flow path are arranged and configured to pump dialysate through the dialysate-side of the dialyzer. To this end, the HD machine may include, inter alia, first and second dialysate pumps to circulate dialysate through the dialysate path. In addition, the HD machine may include a sorbent cartridge for regenerating used or spent dialysate after it leaves the dialyzer. In use, the first and second pumps may move the dialysate through the sorbent cartridge and into a dialysate reservoir, out of dialysate reservoir, and back through the dialyzer.

The extracorporeal blood path includes a to-patient connector such as, for example, a needle, at the end of a venous return line, a from-patient connector such as, for example, a needle, at an end of an arterial line, and a blood pump arranged and configured to pump or pull blood from the patient through the arterial line through the blood side of the dialyzer and back to the patient through the venous return line.

In use, the dialyzer includes a cartridge comprising a semi-permeable membrane (e.g., a high flux membrane) arranged and configured to remove toxins from the blood. In one embodiment, the removal of toxins is accomplished by establishing a concentration gradient across the semi-permeable membrane by allowing the dialysate to flow on one side of the membrane in one direction while simultaneously allowing the blood to flow on the other side of the membrane in the opposite direction. The dialyzer may be of any type suitable for hemodialysis, hemodiafiltration, hemofiltration, or hemoconcentration, as are known in the art.

It should be appreciated that while a particular embodiment of a hemodialysis system including a dialysis flow path, an extracorporeal blood flow path, and a dialyzer have been described, any number of different hemodialysis systems now known or hereafter developed may be utilized. Further, the system described herein may be utilized in connection with other types of dialysis treatments, including, for example, hemofiltration, hemodiafiltration and/or PD systems.

Generally speaking, many dialysis treatments, especially HD, occur in outpatient treatment centers, rooms, facilities, or the like (terms used interchangeably herein without the intent to limit or distinguish). These treatment centers may be located within nursing homes, hospitals, Transitional Care Units (“TCUs”), stand-alone facilitates, etc. A common problem with these treatment centers is the initial construction cost and time for setting up the treatment centers. For example, these treatment centers require, inter alia, a dialysis den, which includes all of the necessary plumbing needed to discard effluent, fresh water used to flush and clean the HD system and/or to mix the dialysate, and any needed electrical power supply to perform the dialysis treatment. As a result, construction of dialysis dens is time-consuming and costly.

That is, in a HD treatment, effluent must be discarded into a waste drain. However, current systems only enable the drain tube to extend for short runs (e.g., 10 feet). As a result, the dialysis treatment needs to be performed in relatively close proximity to the waste drain. In addition, the drain tube relies on gravity to move (e.g., transport) the effluent and thus cannot rise vertically. As a result, generally speaking, each dialysis den includes modular chases (e.g., a designated area for receiving utilities), connection points for each patient's station, multiple waste drains for receiving the effluent, multiple water supply lines, etc. Each waste drain being constructed within the floor (e.g., concrete floor) of the dialysis den. Thus, dialysis dens often include multiple beds positioned adjacent to a waste drain constructed within a concrete floor.

It would be beneficial to provide a system that facilitates easier discarding of effluent. In addition, it would be beneficial to provide a device or system that reduces the initial construction time and cost associated with building dialysis dens. Moreover, it would be beneficial to provide a mobile system thus enabling the system to be easily removed if warranted. It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

According to an exemplary embodiment of the present disclosure, a mobile interface cart is disclosed. In some embodiments, the mobile interface cart includes, inter alia, a storage tank for receiving effluent, a pump for pumping (e.g., moving, transferring, etc.) the effluent to a waste drain, a fresh water connector arranged and configured to connect to a water supply line for enabling fresh water hookup, and an electrical connector arranged and configured to receive electrical power. In some embodiments, the mobile interface cart includes wheels for enabling the mobile interface cart to be easily moved. In some embodiments, the mobile interface cart includes a storage area for storing dialysate containers.

In one example, a mobile interface cart arranged and configured to receive a dialysis machine is disclosed. The mobile interface cart including a storage tank arranged and configured to receive effluent from the dialysis machine, a pump in fluid communication with the storage tank, an electrical inlet for receiving electrical power and to supply power to the pump, a water inlet for receiving water, and a drain outlet arranged and configured to connect to tubing to transfer effluent to an external waste drain. The pump bring arranged and configured to pump effluent from the storage tank to the external waste drain through the drain outlet.

In any preceding or subsequent example, the mobile interface cart includes a connection box, the connection box including the electrical inlet, the water inlet, and the drain outlet.

In any preceding or subsequent example, the connection box is formed in a rear surface of the mobile interface cart.

In any preceding or subsequent example, the mobile interface cart further includes an electrical connector arranged and configured to receive power from the electrical inlet and to supply power to the dialysis machine, a water connector arranged and configured to receive water from the water inlet and to supply water, and a drain connector arranged and configured to receive effluent via the dialysis machine and to transfer effluent to the storage tank.

In any preceding or subsequent example, the mobile interface cart includes a front portion and a rear portion, the front portion including a front height, the rear portion including a rear height, the rear height being greater than the front height to define an intermediate, front-facing surface.

In any preceding or subsequent example, the rear portion includes an enclosure defining an interior space and including a door arranged and configured to provide selective access to the interior space, the interior space housing the pump and the storage tank.

In any preceding or subsequent example, the electrical connector, the water connector, and the drain connector are positioned within the intermediate, front-facing surface.

In any preceding or subsequent example, the front portion of the mobile interface cart includes a top surface arranged and configured to receive the dialysis machine thereon, the HD machine being electrically coupled to the electrical connector positioned in the intermediate, front-facing surface.

In any preceding or subsequent example, the mobile interface cart further includes a water box, the water box including the water connector and the drain connector, the water box formed in the intermediate, front-facing surface.

In any preceding or subsequent example, the storage tank includes a sensor system including a first sensor arranged and configured to detect a first effluent level and a second sensor arranged and configured to detect a second effluent level, the second effluent level being greater than the first effluent level.

In any preceding or subsequent example, the pump is configured to activate when the effluent in the storage tank reaches the first effluent level, and an alarm is generated when the effluent reaches the second effluent level.

In any preceding or subsequent example, the mobile interface cart further includes a cavity arranged and configured to store dialysate.

In any preceding or subsequent example, the mobile interface cart includes a plurality of wheels.

In an alternate example, a dialysis system is disclosed. The dialysis system including a dialysis machine arranged and configured to perform hemodialysis on a patient, and a mobile interface cart, the dialysis machine positioned on the mobile interface cart. The mobile interface cart including a storage tank arranged and configured to receive effluent from the dialysis machine, a pump in fluid communication with the storage tank, an electrical inlet for receiving electrical power and to supply power to the pump, a water inlet for receiving water, and a drain outlet arranged and configured to connect to tubing to transfer effluent to an external waste drain. The pump being arranged and configured to pump effluent from the storage tank to the external waste drain through the drain outlet.

In any preceding or subsequent example, the mobile interface cart includes a connection box, the connection box including the electrical inlet, the water inlet, and the drain outlet.

In any preceding or subsequent example, the connection box is formed in a rear surface of the mobile interface cart.

In any preceding or subsequent example, the mobile interface cart further includes an electrical connector arranged and configured to receive power from the electrical inlet and to supply power to the dialysis machine, a water connector arranged and configured to receive water from the water inlet and to supply water, and a drain connector arranged and configured to receive effluent via the dialysis machine and to transfer effluent to the storage tank.

In any preceding or subsequent example, the mobile interface cart includes a front portion and a rear portion, the front portion including a front height, the rear portion including a rear height, the rear height being greater than the front height to define an intermediate, front-facing surface.

In any preceding or subsequent example, the rear portion includes an enclosure defining an interior space and including a door arranged and configured to provide selective access to interior space, the interior space housing the pump and storage tank.

In any preceding or subsequent example, the electrical connector, the water connector, and the drain connector are positioned within the intermediate, front-facing surface.

In any preceding or subsequent example, the front portion of the mobile interface cart includes a top surface arranged and configured to receive the dialysis machine thereon, the dialysis machine being electrically coupled to the electrical connector positioned in the intermediate, front-facing surface.

In any preceding or subsequent example, the mobile interface cart further includes a cavity arranged and configured to store dialysate.

In any preceding or subsequent example, the mobile interface cart includes a plurality of wheels.

### BRIEF DESCRIPTION OF THE DRAWINGS

By way of example, specific embodiments of the disclosed methods and devices will now be described, with reference to the accompanying drawings, in which:

FIG. 1 illustrates an example of an embodiment of a mobile interface cart in accordance with one or more features of the present disclosure, the mobile interface cart illustrated with an example of an embodiment of a hemodialysis machine and a water and dialysate preparation device;

FIG. 2 illustrates a front perspective view of the mobile interface cart shown in FIG. 1, the mobile interface cart illustrated with a hemodialysis machine and with portions of the cart transparent;

FIG. 3 illustrates a front perspective view of the mobile interface cart shown in FIG. 1;

FIG. 4 illustrates an alternate front perspective view of the mobile interface cart shown in FIG. 1;

FIG. 5 illustrates a rear perspective view of the mobile interface cart shown in FIG. 1;

FIG. 6 illustrates a front view of the mobile interface cart shown in FIG. 1;

FIG. 7 illustrates a rear view of the mobile interface cart shown in FIG. 1;

FIG. 8 illustrates a top view of the mobile interface cart shown in FIG. 1;

FIG. 9 illustrates a bottom view of the mobile interface cart shown in FIG. 1;

FIG. 10 illustrates a front perspective view of the mobile interface cart shown in FIG. 1, the mobile interface cart illustrated with portions of the cart transparent;

FIG. 11 illustrates a rear perspective view of the mobile interface cart shown in FIG. 1, the mobile interface cart illustrated with portions of the cart transparent;

FIG. 12 illustrates a front view of the mobile interface cart shown in FIG. 1, the mobile interface cart illustrated with portions of the cart transparent;

FIG. 13 illustrates a rear view of the mobile interface cart shown in FIG. 1, the mobile interface cart illustrated with portions of the cart transparent;

FIG. 14 illustrates a side view of the mobile interface cart shown in FIG. 1, the mobile interface cart illustrated with portions of the cart transparent;

FIG. 15 illustrates a rear perspective view of the mobile interface cart shown in FIG. 1; and

FIG. 16 illustrates a detailed view of an example of an embodiment of a water box positioned within the mobile interface cart shown in FIG. 1.

### DETAILED DESCRIPTION

The present embodiments will now be described more fully hereinafter with reference to the accompanying drawings, in which several exemplary embodiments are shown. The subject matter of the present disclosure, however, may be embodied in many different forms and types of devices and systems for dialysis and other potential medical devices and treatments, and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

Exemplary embodiments of a mobile interface cart used in connection with a dialysis system are disclosed herein. In use, the mobile interface cart is arranged and configured to be used with a dialysis machine, such as, for example, an HD machine or in an HD system. In accordance with one or more features of the present disclosure, the HD machine may be connected to the mobile interface cart via, for example, tubing, so that the used effluent from the HD machine may be transferred to a waste drain via the mobile interface cart. That is, in some embodiments, the mobile interface cart includes an effluent storage tank and a pump. In use, the effluent is transferred to the effluent storage tank. Thereafter, the pump may be activated to transfer or discard the effluent from the effluent storage tank to the waste drain located within the treatment center. Thus arranged, by utilizing a pump and a storage tank positioned within the mobile interface cart, increased flexibility and access to waste drains in the treatment center is provided, which reduces the initial construction cost and time associated with constructing waste drains in the concrete floor of the dialysis dens. In use, the mobile interface cart incorporating the pump enables the effluent to be transferred much greater distances and/or over and around obstacles (e.g., the pump enables the effluent to be transferred from the effluent storage tank to the waste drain over much longer runs thereby eliminating, or at least greatly minimizing, the need for the HD machine to be positioned adjacent or in close proximity to a waste drain).

In addition, in accordance with one or more features of the present disclosure, the mobile interface cart provides a self-contained device that facilitates easy repositioning of dialysis equipment. That is, for example, the mobile interface cart provides a self-contained device that enables treatment centers to easily reposition equipment as needed. In some embodiments, the mobile interface cart can be repositioned as needed. Once positioned, the mobile interface cart merely needs to be connected to (e.g., supplied with) electrical power, fresh water, and access to a waste drain.

While the mobile interface cart will be shown and described in the present disclosure for use with one or more dialysis machines, it should be appreciated that the mobile interface cart should not be limited to any particular type of dialysis machine. It is envisioned that the mobile interface cart according to one or more features of the present disclosure may be used in connection with other types of dialysis machines. Thus, the present disclosure should not be limited to any particular type of dialysis machine unless specifically claimed.

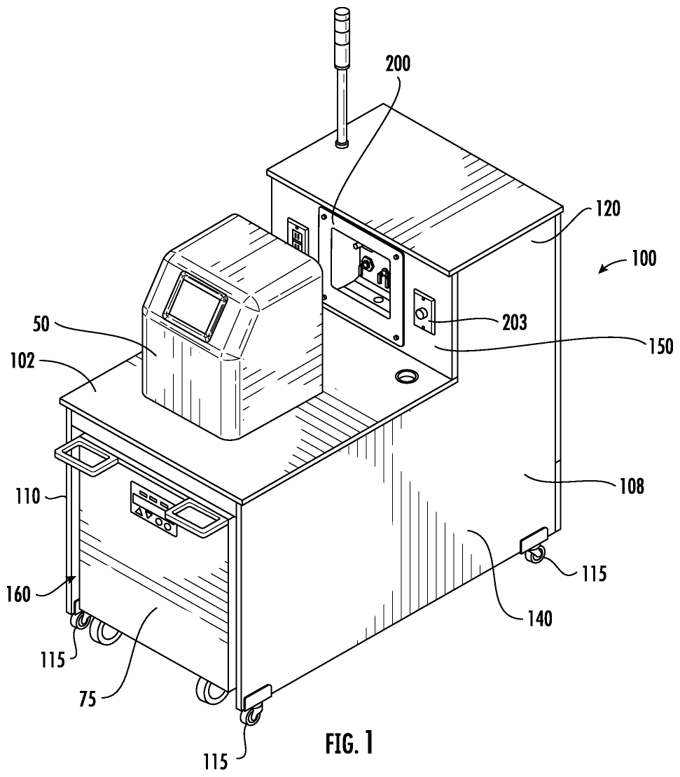

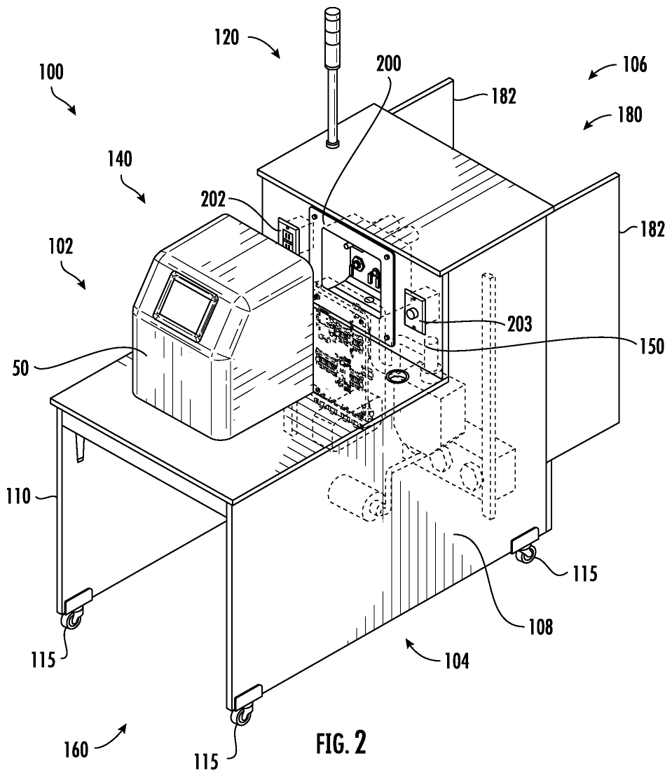

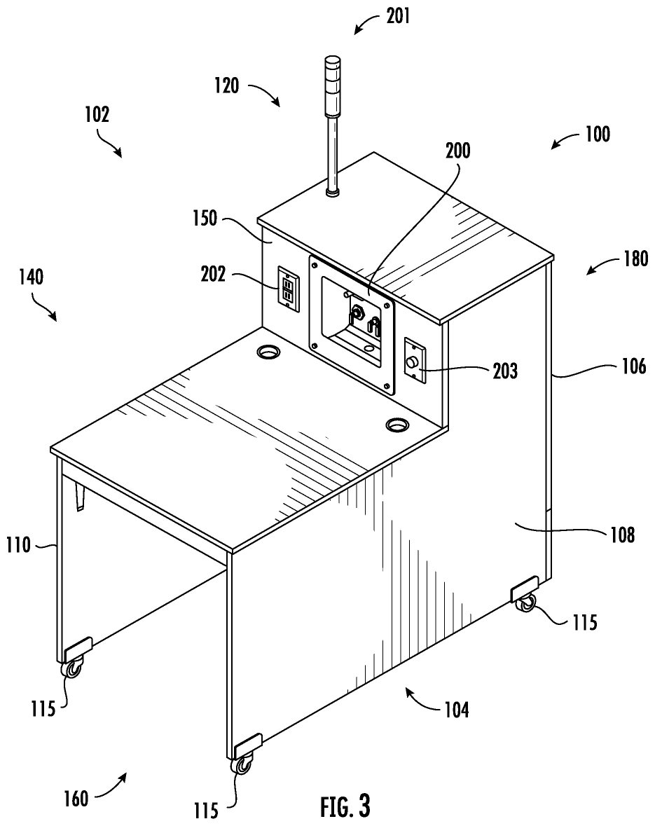

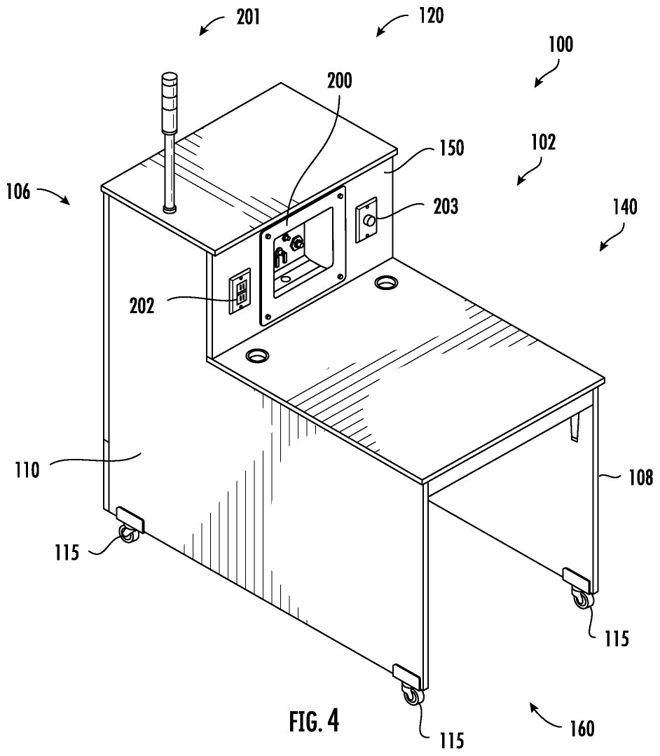

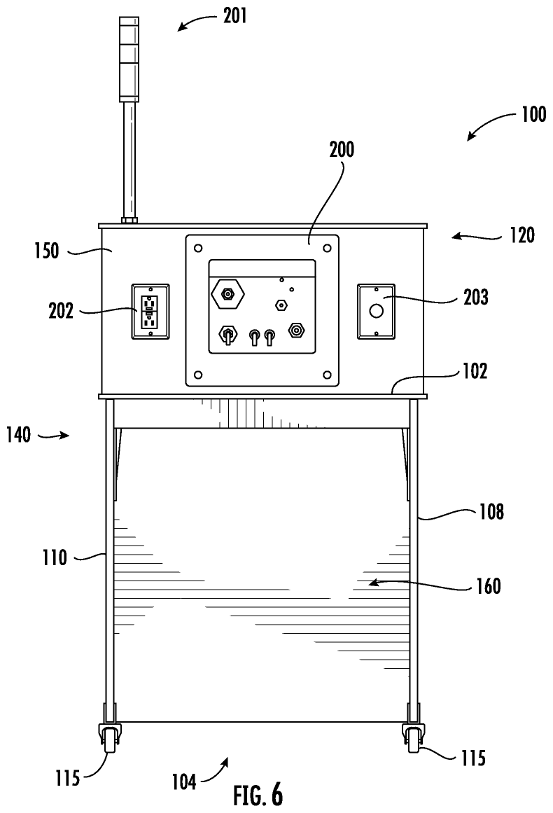

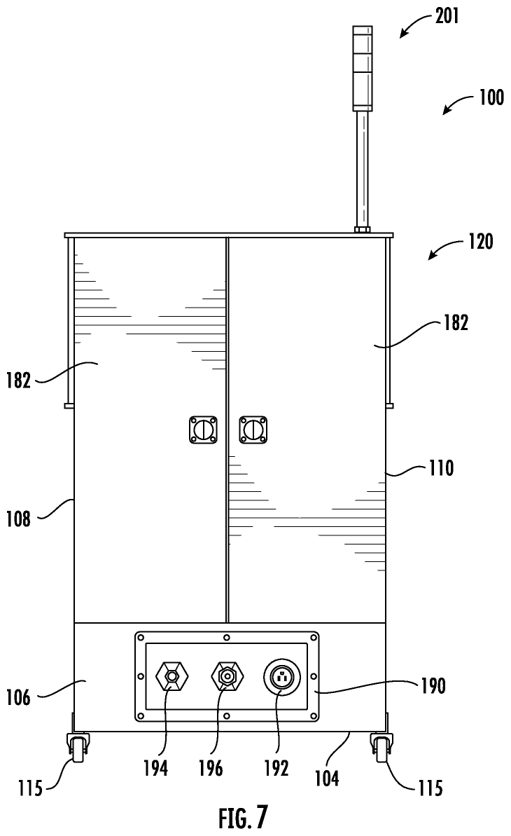

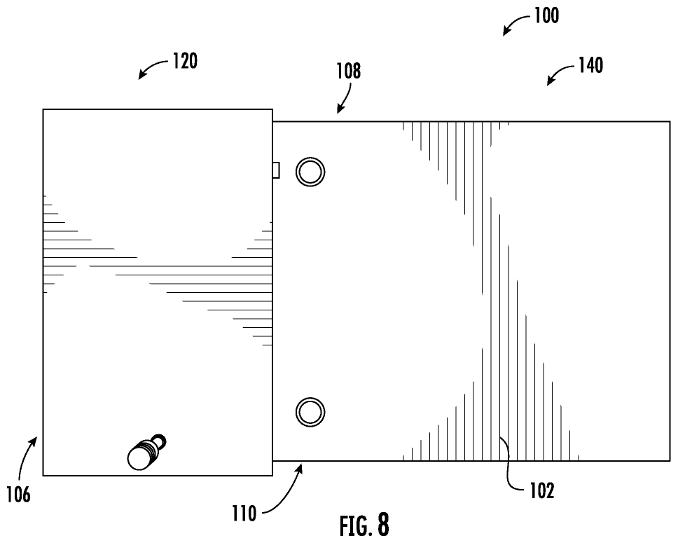

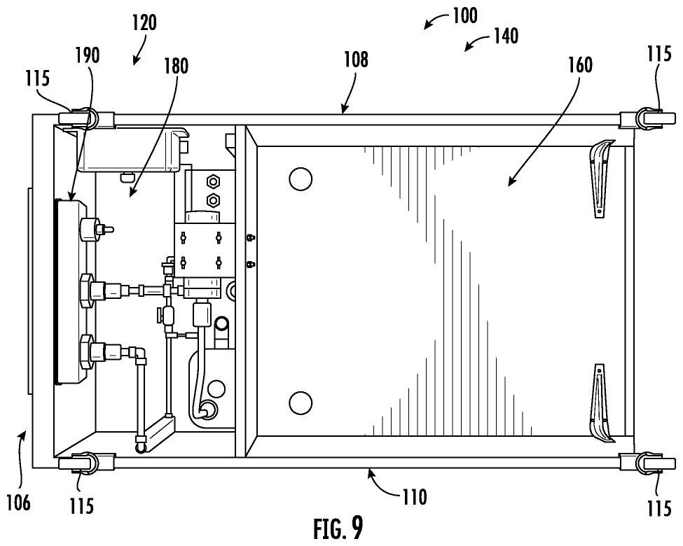

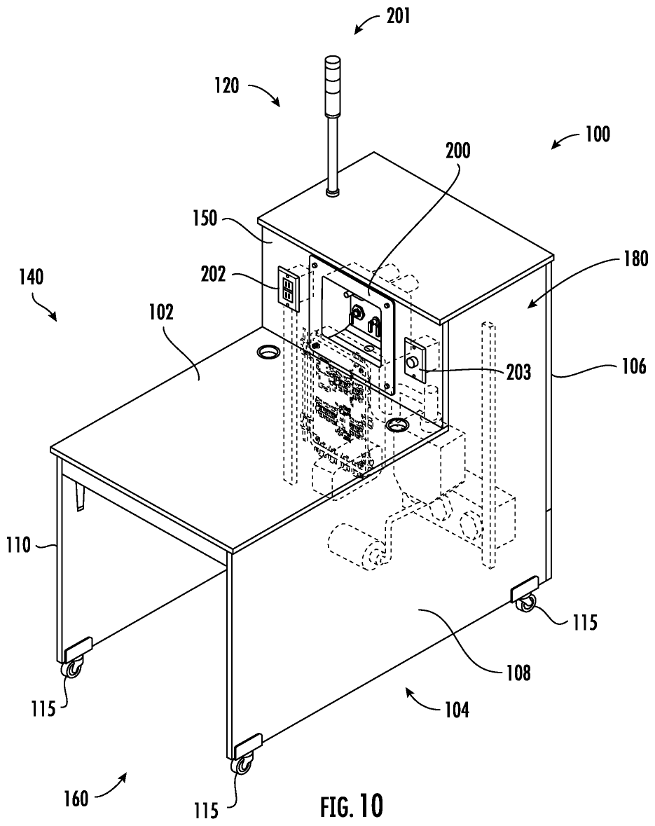

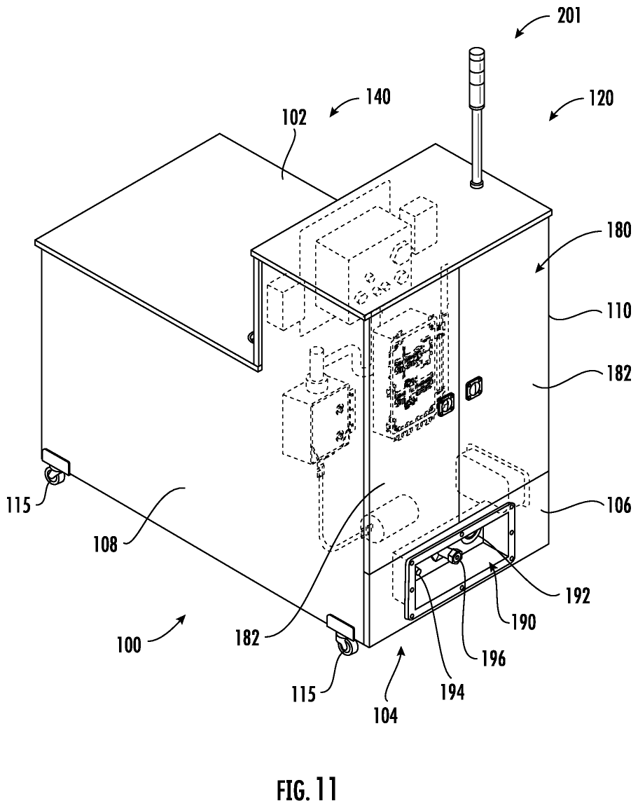

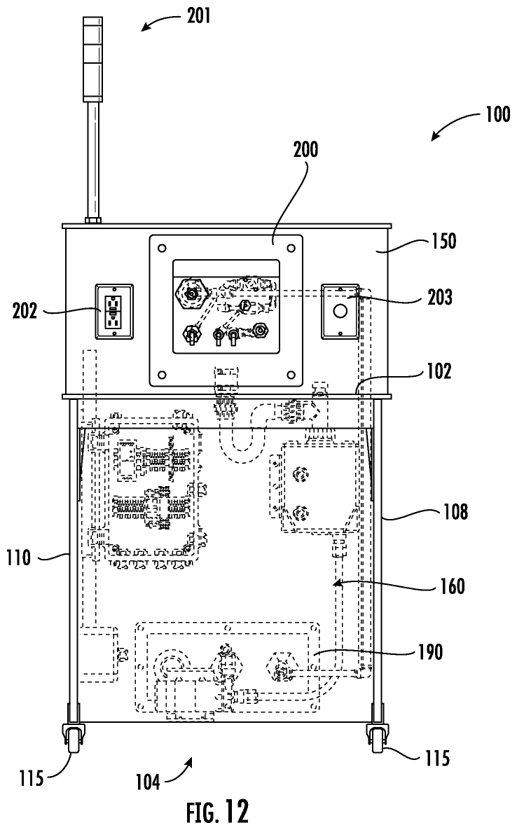

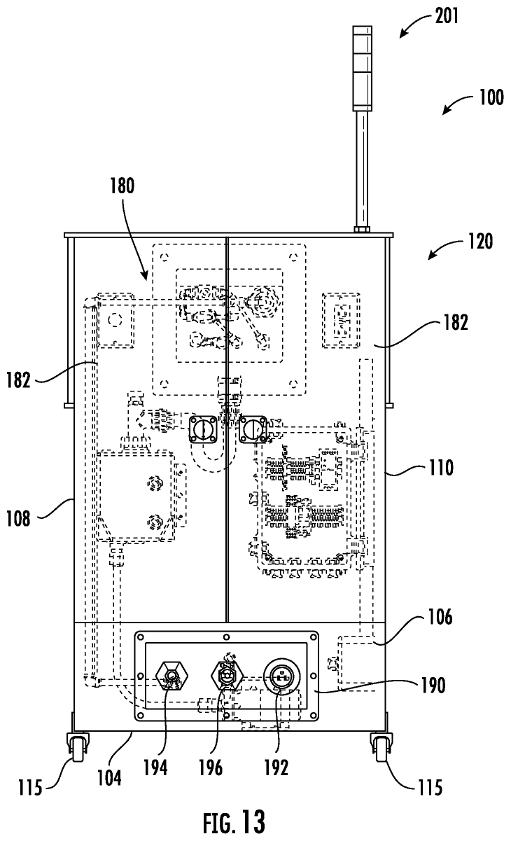

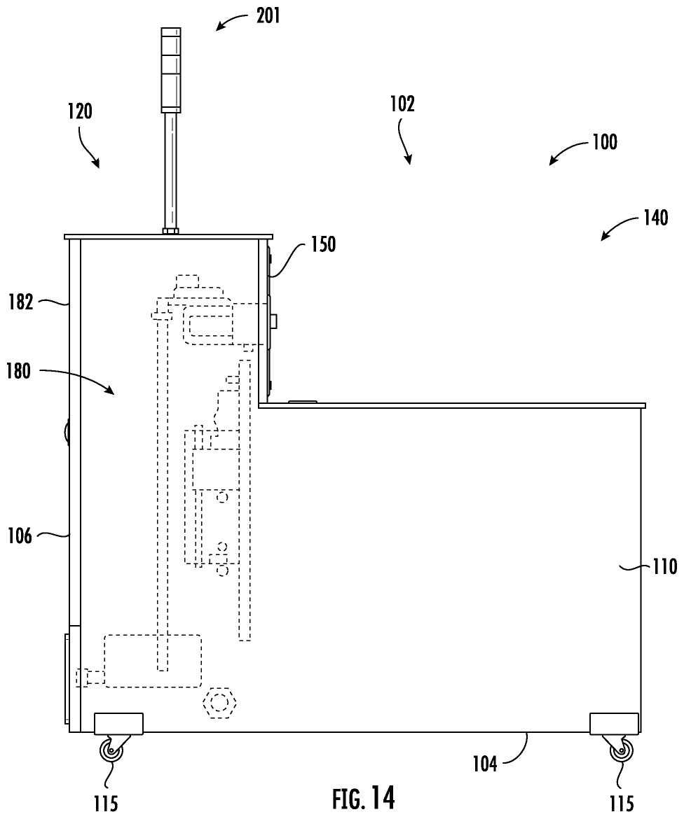

Referring to FIGS. 1-16, an example of an embodiment of a mobile interface cart 100 is illustrated. Generally speaking, as illustrated, the mobile interface cart 100 includes a top surface 102, a bottom surface 104, a rear surface 106, and side surfaces 108, 110. In some embodiments, as illustrated, the mobile interface cart 100 may include a rear portion 120 and a front portion 140. As illustrated, the rear portion 120 may define a rear height and the front portion 140 may define a front height, the rear height being greater than the front height. Thus arranged, the mobile interface cart 100, and more specifically the rear portion 120, may include an intermediate, front facing surface 150. As will be described in greater detail below, a water box 200 and other components may be positioned within the intermediate, front facing surface 150. In use, an HD machine 50, such as, for example, a NxStage System One hemodialysis machine manufactured by Fresenius Medical Care, may be fluidly coupled via tubing to the water box 200. As illustrated, the mobile interface cart 100 may include a plurality of wheels 115 to easy repositioning of the mobile interface cart 100.

In use, the mobile interface cart 100 is arranged and configured to receive the HD machine 50. For example, as illustrated, the mobile interface cart 100 includes a surface for positioning of the HD machine 50 thereon. In some embodiments, the top surface 102 of the front portion 140 may define the surface for receiving the HD machine 50 thereon. Thus arranged, the HD machine 50 may be positioned on the top surface 102 of the front portion 140 and be connected to the water box 200 and other components positioned within the intermediate, front facing surface 150, although this is but one configuration.

In addition, in some embodiments, the front portion 140 may include a cavity, a garage, an opening, or the like 160 (terms used interchangeably herein without the intent to limit or distinguish). In use, the cavity 160 is arranged and configured to receive and store, for example, the dialysate or dialysis solution (terms used interchangeably herein without the intent to limit or distinguish). For example, as illustrated in FIG. 1, in some embodiments, the cavity 160 formed in the front portion 140 of the mobile interface cart 100 may receive a water and dialysate preparation device 75, such as, for example, a Pure-Flow™ water and dialysate preparation device manufactured by Fresenius Medical Care, which is arranged and configured to mix and store the needed dialysis. Alternatively, in alternate embodiments, the cavity 160 may be arranged and configured to store, for example, pre-mixed dialysis containers or bags (e.g., the cavity 160 may be equipped with hooks, shelves, etc. to store, hang, etc. the dialysis bags or containers). Alternatively, the cavity 160 formed in the front portion 140 of the mobile interface cart 100 may be both arranged and configured to selectively receive the water and dialysate preparation device 75 and the pre-mixed dialysis containers or bags thereby providing an end user with additional options and flexibility.

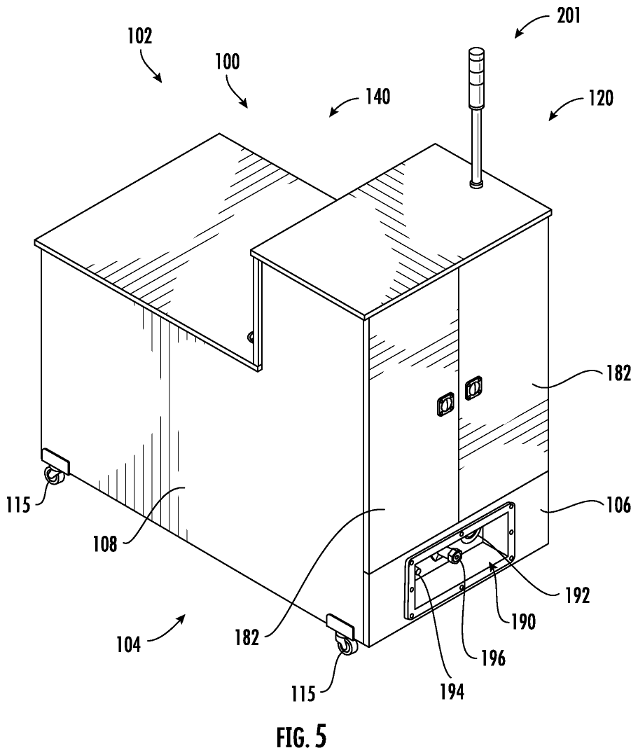

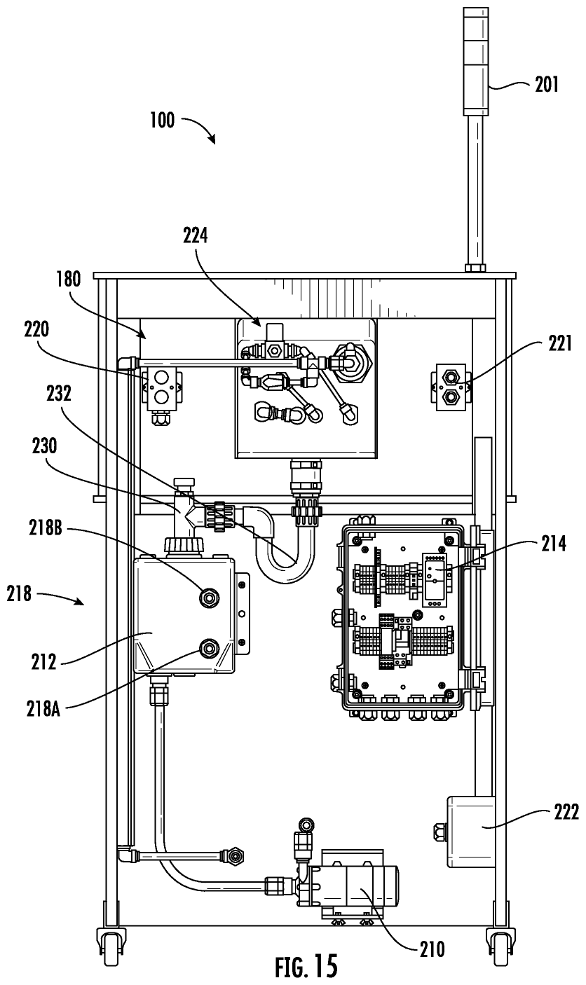

The rear portion 120 of the mobile interface cart 100 may include an enclosure, a cabinet, or the like 180 (terms used interchangeably herein without the intent to limit or distinguish). As illustrated, the enclosure 180 may include a door 182 to facilitate opening and closing to selectively enable access to the interior space or cavity of the enclosure 180. With reference to FIGS. 5, 7, 11, and 13, the rear surface 106 includes a connection box or a utilities interface box 190 (terms used interchangeably herein without the intent to limit or distinguish). As illustrated, and as will be described in greater detail below, the connection box 190 includes an electrical connection 192 (e.g., an electrical inlet) for coupling the mobile interface cart 100 to electrical power, a water connection 194 (e.g., a water inlet) for coupling the mobile interface cart 100 to water, and a drain connection 196 (e.g., a drain outlet) for coupling the mobile interface cart 100 to an external drainage point or external waste drain (terms used interchangeably herein without the intent to limit or distinguish). Thus arranged, the mobile interface cart 100 includes, inter alia, an electrical connection (e.g., an electrical inlet) 192, a water connection (e.g., a water inlet) 194, and a drain connection (e.g., a drain outlet) 196 for coupling the mobile interface cart 100 to all necessary utilities (e.g., power, water, and drain). In addition, as best illustrated in FIG. 15 and as will be described in greater detail below, positioned within the interior cavity of the enclosure 180 is a pump 210, a storage tank 212, and an electrical box or control panel 214 (terms used interchangeably herein without the intent to limit or distinguish). In use, the electrical connection (e.g., electrical inlet) 192, the water connection (e.g., water inlet) 194, and the drain connection (e.g., drain outlet) 196 may be configured in any suitable form now know or hereafter developed.

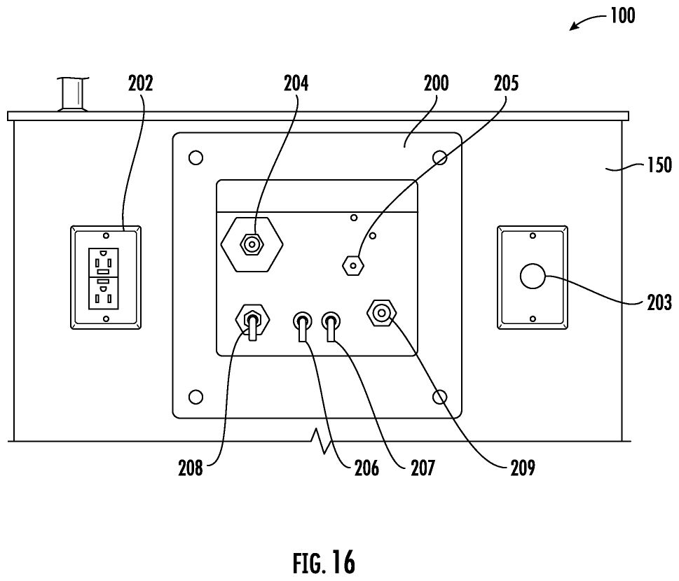

As previously mentioned, with reference to FIG. 16, in some embodiments, the mobile interface cart 100, and more specifically, the intermediate, front-facing surface 150, may include, inter alia, a water box 200 and other components such as, for example, an electrical connector or outlet 202. In use, the electrical outlet 202 is electrically coupled to the electrical box or control panel 214, which is electrically coupled to the electrical connection 192. As such, the HD machine 50 positioned on the top surface 102 of the front portion 140 of the mobile interface cart 100 may be electrically coupled (e.g., plugged into) the electrical outlet 202 to supply line-voltage power to power the HD machine 50. In addition, the electrical outlet 202 and/or the electrical box or control panel 214 may be used to supply power to the optional water and dialysate preparation device 75 when used and any other components of the mobile interface cart 100 requiring power. In use, the electrical outlet 202 may be any suitable outlet now known or hereafter developed such as, a ground fault circuit interrupter (GFCI).

In addition, the water box 200 includes one or more fresh water supply ports or connectors 204 for supplying water to, for example, the water and dialysate preparation device 75 and/or to supply fresh water for flushing and cleaning the HD system and tubes. For example, in some embodiments, as illustrated, the water box 200 includes a fresh water supply port 204 for supplying water to the water and dialysate preparation device 75 when used. In use, the fresh water supply port 204 is fluidly coupled to the water connection 194 for supplying fresh water. In use, the fresh water supply port 204 may be any suitable connector now known or hereafter developed such as, for example, a male quick disconnector.

In addition, the water box 200 includes one or more drain ports or connectors to connect to the various components to transfer the effluent and/or fresh water. For example, the water box 200 may include a drain coupling input port 205 to facilitate draining of the dialysis bags or containers with the output of the bags or containers diverting from input port 205 through output port 206 to drain, when the optional water and dialysate preparation device 75 is not used. In addition, the water box 200 may include a drain coupling input 209 for coupling from the optional water and dialysate preparation device 75 drain output, and when used, the output will be diverted through output port 207 to drain. In addition, the water box 200 may include a domestic water output port 208, which is utilized during flushing of the system when switch 203 is activated as will be described in greater detail below.

Thus arranged, the water box 200 includes one or more water connectors or connections and drain connectors or connections to connect to the various components including the HD machine 50 and the optional water and dialysate preparation device 75 or the dialysis bags or containers, etc. As such, the water box 200 connects the HD machine 50 and the water and dialysate preparation device 75 or dialysis bags or containers to the connection box 190 and to the storage tank 212 and pump 210 to transfer and drain fluid as needed. In use, the water box 200, and more specifically, the drain ports positioned within the water box 200, are in fluid connection with the storage tank 212, which is in fluid connection with the pump 210 via, for example, tubing. In addition, the pump 210 is in fluid connection via tubing with the drain connection 196. As such, in use, the HD machine 50 may be fluidly coupled to the drain ports in the water box 200. Thus arranged, the effluent from the HD process may be pumped by the HD machine 50 into the storage tank 212. Subsequently, the effluent may be pumped from the storage tank 212 to the external waste drain located within the treatment center via tubing connected to the drain connection 196.

In accordance with one or more features of the present disclosure, by incorporating the pump 210 into the mobile interface cart 100, increased flexibility to access the external waste drain in the treatment center is enabled. That is, in contrast to systems in which the HD machine is positioned adjacent to, or in close proximity with, the external waste drain formed in the floor of the treatment center, the mobile interface cart 100 enables the effluent to be pumped via tubing to the external waste drain located some distance from the HD machine. In addition, in contrast to systems which rely on gravity to move the effluent, the mobile interface cart 100 enables the effluent to be pumped via tubing up, over, around, etc. obstacles such as, for example, windows, doors, etc. That is, in use, by utilizing the mobile interface cart 100 with a pump 210, the effluent can be pumped via tubing to distant external waste drains positioned within the treatment centers thereby eliminating the need to install expensive waste drains in the floor of the treatment centers adjacent to the HD machines (e.g., the mobile interface cart 100 and the pump 210 enable the tubing to run longer distances and around obstacles providing significant benefits to the owner of the treatment centers). In some embodiments, it has been found that the effluent can be pumped vertically by a distance of ten (10) feet or more and effluent can be pumped horizontally by a distance of twenty (2), thirty (30), or more feet.

In some embodiments, the mobile interface cart 100 may also include a shutoff level to turn OFF the water supply, if needed, an alarm 201, for example, a visual alarm, to provide a visual indication that the pump 210 has failed as will be described in greater detail below, and a switch 203, such as, for example, a manual switch such as, a momentary push button switch, although other switches may be used, to flush the system and tubes, etc. (e.g., to run fresh water directly from the water connection 194 through the tubing). That is, in use, activation of the switch 203 initiates a wall box flush with fresh water (e.g., water received from the water connection 194 (e.g., a water inlet) is moved via the interconnecting tubing through the water box 200 to the storage tank 212 and pump 210 and out through the drain connection 196 (e.g., drain outlet) to the external waste drain in the treatment center. As such, the tubing and other connected components can be cleaned.

With reference to FIG. 15, in some embodiments, the storage tank 212 may include a sensor system 218 arranged and configured to determine, measure, etc. a quantity of the fluid, effluent, etc. in the storage tank 212. As illustrated, in some embodiments, the sensor system 218 may include a lower or first sensor 218A and an upper or second sensor 218B. In use, the pump 210 is arranged and configured to begin pumping fluid or effluent when the level of fluid or effluent in the storage tank 212 reaches the first or lower sensor 218A. Thus arranged, the pump 210 is not activated until a predetermined amount of fluid or effluent is positioned within the storage tank 212. In the instance where the fluid or effluent reaches the level of the second or upper sensor 218B, an alarm such as, for example, alarm 201, may be triggered indicating that the pump 210 may have failed. For example, in the illustrated example embodiment, the alarm 201 may be configured as a probe or a light tower extending from the top surface of the mobile interface cart 100. In use, the probe or light tower may include, for example, a green light and a red light. In use, the green light may be illuminated when the mobile interface cart 100 receives power and is operating normally. The red light may be illuminated when the fluid or effluent reaches the level of the second or upper sensor 218B indicating that the pump 210 is not operating properly and the storage tank 212 is not being emptied. In addition, and/or alternatively, an audio alarm may be triggered. Moreover, the sensor system 218 may be configured to transmit a signal to remote devices such as, for example a central station positioned within the treatment center.

In addition, with reference to FIG. 15, the interior cavity of the enclosure 180 may include any additional components needed to operate the mobile interface cart 100. For example, a junction box 220 for housing the switch 203, a junction box 221 for housing the electrical outlet 202 and for continuing power from the electrical box or control panel 214, a junction box 222 for interfacing with the electrical connection 192 and for continuing power to the electrical box or control panel 214, and a solenoid valve 224 for operating the flush when the switch 203 is activated. In addition, the interior cavity of the enclosure 180 may include any additional plumbing needed to operate the mobile interface cart 100. For example, tubing, a vent assembly 230 for venting the storage tank 212, and a plumbing trap 232.

As previously mentioned, the mobile interface cart 100 includes a water connection 194 to supply fresh water to the mobile interface cart 100. In use, if utilized with the water and dialysate preparation device 75, the fresh water can be used to mix the dialysate. In addition, and/or alternatively, the fresh water can be used to flush (e.g., clean) the tubing. For example, in use, the mobile interface cart 100 may include the switch 203 positioned, for example, in the intermediate, front-facing surface 150. Activation of the switch 203 causes fresh water received through the water connection 194 to run through the tubing to flush and clean the tubing.

In use, the electrical box or control panel 214 is electrical coupled to the electrical connection 192 for receiving line-voltage power. In addition, the electrical box or control panel 214 is electrically coupled to any other components located in the mobile interface cart 100 to supply power as needed including, for example, the pump 210, the electrical outlet 202, the sensor system 218, etc. In addition, the electrical box or control panel 214 may include any necessary circuitry, processors, etc. needed to operate the components of the mobile interface cart 100.

Thus arranged, by incorporating one or more features of the mobile interface cart 100, the effluent from the HD dialysis procedure can be readily discarded without the need to construct expensive dens including multiple waste drains. Rather, in use, the mobile interface cart 100 can be positioned as needed in a treatment center. Thereafter, the mobile interface cart can be coupled to electrical and water supply lines. In addition, tubing can be coupled to the drainage access port, the tubing extending to an external waste drain positioned somewhere in the treatment center. As such, the need for expensive and time-consuming waste drains to be constructed within the floors adjacent to each HD machine is eliminated.

The mobile interface cart described herein has been explained in connection with dialysis machines and/or systems having a particular configuration. It is contemplated that the mobile interface cart described herein may be used with dialysis machines and/or systems having other configurations, for example, different types of dialysis machines. The s mobile interface cart described herein may be used with any appropriate dialysis machine and/or system.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. In addition, while certain embodiments have been described and illustrated with certain features, it is envisioned that features of one embodiment may be used in combination with other embodiments. Moreover, the following claims are hereby incorporated into this Detailed Description by this reference, with each claim standing on its own as a separate embodiment of the present disclosure.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A mobile interface cart arranged and configured to receive a dialysis machine, the mobile interface cart comprising:
a storage tank arranged and configured to receive effluent from the dialysis machine;
a pump in fluid communication with the storage tank;
an electrical inlet for receiving electrical power and to supply power to the pump;
a water inlet for receiving water; and
a drain outlet arranged and configured to connect to tubing to transfer effluent to an external waste drain;
wherein the pump is arranged and configured to pump effluent from the storage tank to the external waste drain through the drain outlet.

2. The mobile interface cart of claim 1, wherein the mobile interface cart includes a connection box, the connection box including the electrical inlet, the water inlet, and the drain outlet.

3. The mobile interface cart of claim 2, wherein the connection box is formed in a rear surface of the mobile interface cart.

4. The mobile interface cart of claim 1, further comprising:
an electrical connector arranged and configured to receive power from the electrical inlet and to supply power to the dialysis machine;
a water connector arranged and configured to receive water from the water inlet and to supply water; and
a drain connector arranged and configured to receive effluent via the dialysis machine and to transfer effluent to the storage tank.

5. The mobile interface cart of claim 4, wherein the mobile interface cart includes a front portion and a rear portion, the front portion including a front height, the rear portion including a rear height, the rear height being greater than the front height to define an intermediate, front-facing surface.

6. The mobile interface cart of claim 5, wherein the rear portion includes an enclosure defining an interior space and including a door arranged and configured to provide selective access to the interior space, the interior space housing the pump and the storage tank.

7. The mobile interface cart of claim 6, wherein the electrical connector, the water connector, and the drain connector are positioned within the intermediate, front-facing surface.

8. The mobile interface cart of claim 7, wherein the front portion of the mobile interface cart includes a top surface arranged and configured to receive the dialysis machine thereon, the HD machine being electrically coupled to the electrical connector positioned in the intermediate, front-facing surface.

9. The mobile interface cart of claim 7, further comprising a water box, the water box including the water connector and the drain connector, the water box formed in the intermediate, front-facing surface.

10. The mobile interface cart of claim 1, wherein the storage tank includes a sensor system including a first sensor arranged and configured to detect a first effluent level and a second sensor arranged and configured to detect a second effluent level, the second effluent level being greater than the first effluent level.

11. The mobile interface cart of claim 10, wherein the pump is configured to activate when the effluent in the storage tank reaches the first effluent level, and an alarm is generated when the effluent reaches the second effluent level.

12. The mobile interface cart of claim 1, wherein the mobile interface cart further comprises a cavity arranged and configured to store dialysate.

13. The mobile interface cart of claim 1, wherein the mobile interface cart includes a plurality of wheels.

14. A dialysis system comprising:
a dialysis machine arranged and configured to perform hemodialysis on a patient; and
a mobile interface cart, the dialysis machine positioned on the mobile interface cart, the mobile interface cart including:
a storage tank arranged and configured to receive effluent from the dialysis machine;
a pump in fluid communication with the storage tank;
an electrical inlet for receiving electrical power and to supply power to the pump;
a water inlet for receiving water; and
a drain outlet arranged and configured to connect to tubing to transfer effluent to an external waste drain;
wherein the pump is arranged and configured to pump effluent from the storage tank to the external waste drain through the drain outlet.

15. The dialysis system of claim 14, wherein the mobile interface cart includes a connection box, the connection box including the electrical inlet, the water inlet, and the drain outlet.

16. The dialysis system of claim 15, wherein the connection box is formed in a rear surface of the mobile interface cart.

17. The dialysis system of claim 14, wherein the mobile interface cart further comprises:
an electrical connector arranged and configured to receive power from the electrical inlet and to supply power to the dialysis machine;
a water connector arranged and configured to receive water from the water inlet and to supply water; and
a drain connector arranged and configured to receive effluent via the dialysis machine and to transfer effluent to the storage tank.

18. The dialysis system of claim 17, wherein the mobile interface cart includes a front portion and a rear portion, the front portion including a front height, the rear portion including a rear height, the rear height being greater than the front height to define an intermediate, front-facing surface.

19. The dialysis system of claim 18, wherein the rear portion includes an enclosure defining an interior space and including a door arranged and configured to provide selective access to interior space, the interior space housing the pump and storage tank.

20. The dialysis system of claim 19, wherein the electrical connector, the water connector, and the drain connector are positioned within the intermediate, front-facing surface.

21. The dialysis system of claim 20, wherein the front portion of the mobile interface cart includes a top surface arranged and configured to receive the dialysis machine thereon, the dialysis machine being electrically coupled to the electrical connector positioned in the intermediate, front-facing surface.

22. The dialysis system of claim 14, wherein the mobile interface cart further comprises a cavity arranged and configured to store dialysate.

23. The dialysis system of claim 14, wherein the mobile interface cart includes a plurality of wheels.
